(19) United States
(12) Patent Application Publication
(10) Pub. No.: US 2011/0108036 A1
(43) Pub. Date: May 12, 2011

(54) RESPIRATORY MASK

(76) Inventor: Loescher Thomas, Carlsbad, CA (US)

(21) Appl. No.: 12/942,194

(22) Filed: Nov. 9, 2010

(30) Foreign Application Priority Data
Nov. 10, 2009 (CN) 200910221429.2

(51) Int. Cl. A61M 16/06

(52) U.S. Cl. 128/206.22

(57) ABSTRACT

A respiratory mask includes a housing, an air supply tube, and a medium. The housing includes an inner surface, an air-passage opening, and a tubular wall that projects inwardly from the inner surface and that defines a receiving hole aligned and communicated with the air-passage opening. The air supply tube is connected fluidly to the air-passage opening. The medium is disposed in the receiving hole, and is made of a material that can absorb heat and moisture.

### CROSS-REFERENCE TO RELATED APPLICATION

This application claims priority of Chinese Application No. 200910221429.2, filed on Nov. 10, 2009.

### BACKGROUND OF THE INVENTION

1. Field of the Invention

This invention relates to a respiratory mask, and more particularly to a respiratory mask used in high gas flow applications and having a heat and moisture exchange function.

2. Description of the Related Art

Continuous positive airway pressure (CPAP) is an effective way of treating obstructive sleep apnea and low blood oxygen levels associated with pulmonary edema and congestive heart failure. Non Invasive Ventilation (NIV) is an effective way of maintaining pre-morbid blood carbon dioxide levels and with the addition of positive end expiratory pressure (PEEP) low blood oxygen levels associated pulmonary insufficiency. Since a respiratory mask used for these applications are held on the face for a long periods of time, masks used for these applications may have ventilation holes within the mask (CPAP) to prevent the condensation of water from the exhaled gas and to reduce inhaled carbon dioxide or come without ventilation holes (NIV) which cause the condensation of water from the exhaled gas.

Further, in both applications, anhydrous gas is delivered to the mask, the upper and lower respiratory tract needs to warm and add water vapor to the gas causing discomfort and drying of the respiratory mucosa. Hence, some applications utilize a heated humidifier to provide the heat and moisture required. Aside from mounting the flow generator with the heated humidifier, another method employed is that as shown in FIGS. 1 and 2, where an artificial nose 12 is mounted on a respiratory mask 11. The artificial nose 12, also known as a heat and moisture exchanger (HME), includes an air supply tube 121 connected between the respiratory mask 11 and a tube 13 of the flow generator. The air supply tube 121 defines a receiving cavity 122 for receiving a medium 123. The medium 123 may be made of a paper or foamed polyurethane material. Through such a configuration, heat and moisture exhaled by the patient may be absorbed and stored. When air supplied by the flow generator passes through the medium 123, the amount of heat and moisture absorbed by the patient during inhalation may be increased.

However, the aforesaid two methods are expensive, cumbersome or create additional problems for the patient and care giver. Use of the heated humidifier or the artificial nose 12 will only complicate the structure of the whole respiratory system. Moreover, since the artificial nose 12 is disposed externally of the respiratory mask 11 so that the medium 123 is distal from the mouth, an additional amount of “dead space” (amount of gas rebreathed) is created which can be detrimental to the therapy by increasing blood carbon dioxide levels.

### SUMMARY OF THE INVENTION

Therefore, the main object of the present invention is to provide a respiratory mask that can quickly absorb and store heat and moisture of gas exhaled by a patient.

Another object of the present invention is to provide a respiratory mask that can release the stored heat and moisture back into the inhaled gas, reducing the impact of dry gas being delivered to the patient.

The purpose of the present invention and the solution to the conventional technical problems are achieved through employment of the below technical means. According to the disclosure of this invention, a respiratory mask comprises a housing, an air supply tube, and a medium. The housing includes an inner surface, an air-passage opening, and a tubular wall that projects inwardly from the inner surface and that defines a receiving hole aligned and communicated with the air-passage opening. The air supply tube is connected fluidly to the air-passage opening. The medium is disposed in the receiving hole, and is made of a material that can absorb heat and moisture.

Through the aforesaid technical means, the advantages and effectiveness of the respiratory mask according to the present invention reside in the fact that through disposal of the medium within the housing and immediately adjacent to the patient's mouth and nose, heat and moisture from gas exhaled by the patient can be quickly absorbed and stored by the medium, thereby enhancing the effect of storing heat and moisture. Further, gas exhaled by the patient passes through the medium and discharges through the air supply tube to thereby minimize the amount of gas rebreathed inhalation.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a perspective view of a conventional artificial nose mounted on a respiratory mask;

FIG. 2 is an exploded perspective view of the conventional artificial nose;

FIG. 3 is a perspective view of a respiratory mask according to the first preferred embodiment of this invention;

FIG. 4 is an exploded perspective view of the first preferred embodiment;

FIG. 5 is a schematic rear view of the first preferred embodiment;

FIG. 6 is a fragmentary sectional view of the first preferred embodiment, illustrating a connection between housing and an air supply tube;

FIG. 7 is a view similar to FIG. 5, but illustrating a tubular sleeve inserted into a tubular wall of the housing;

FIG. 8 is a fragmentary sectional view of the first preferred embodiment, illustrating a medium disposed inside the tubular sleeve and the tubular sleeve inserted into the tubular wall of the housing;

FIG. 9 is a fragmentary sectional view of an alternative form of the first preferred embodiment, illustrating the medium directly disposed inside the tubular wall;

FIG. 10 is a perspective view of a respiratory mask according to the second preferred embodiment of this invention;

FIG. 11 is an exploded perspective view of the second preferred embodiment;

FIG. 12 is a sectional view of the second preferred embodiment, illustrating how a retainer retains an air supply tube to a mounting flange of a shroud;

FIG. 13 is a schematic rear view of the second preferred embodiment, illustrating a tubular sleeve inserted into a tubular wall of the shroud;

FIG. 14 is a fragmentary sectional view of the second preferred embodiment, illustrating a medium disposed inside the tubular sleeve and the tubular sleeve inserted into the tubular wall; and

FIG. 15 is a fragmentary sectional view of an alternative form of the second preferred embodiment, illustrating the medium directly disposed inside the tubular wall.

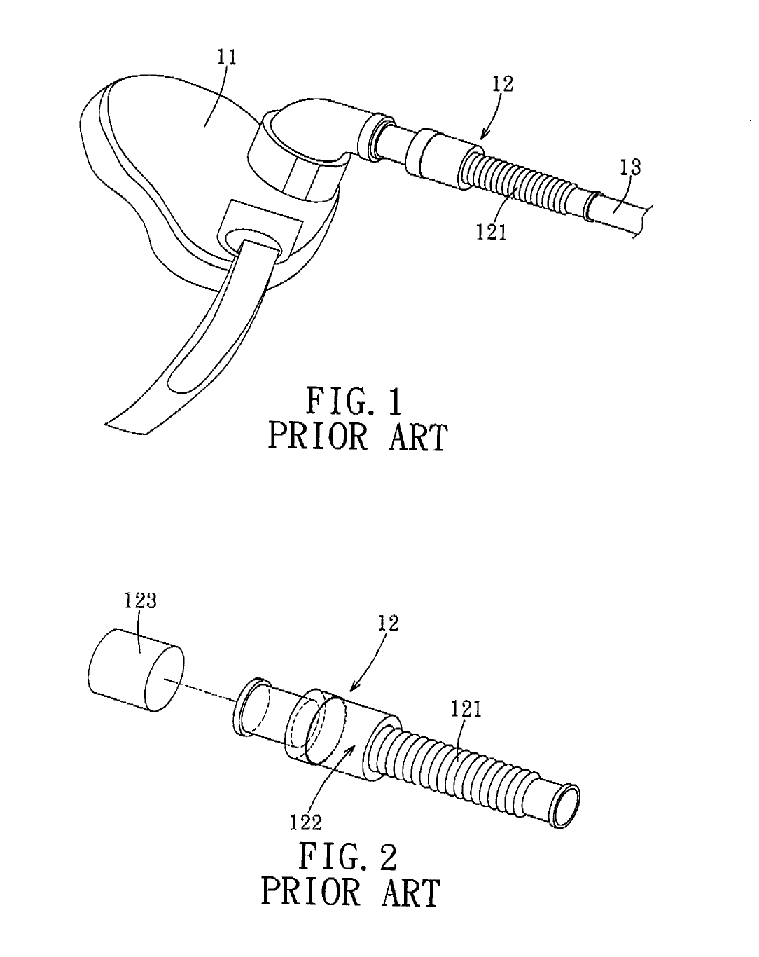

### DETAILED DESCRIPTION OF THE PREFERRED EMBODIMENTS

The above-mentioned and other technical contents, features, and effects of this invention will be clearly presented from the following detailed description of two preferred embodiments in coordination with the reference drawings. Through description of the concrete implementation method, the technical means employed and the effectiveness to achieve the predetermined purposes of the present invention will be thoroughly and concretely understood. However, the enclosed drawings are used for reference and description only, and are not used for limiting the present invention.

Before this invention is described in detail, it should be noted that, in the following description, similar elements are designated by the same reference numerals.

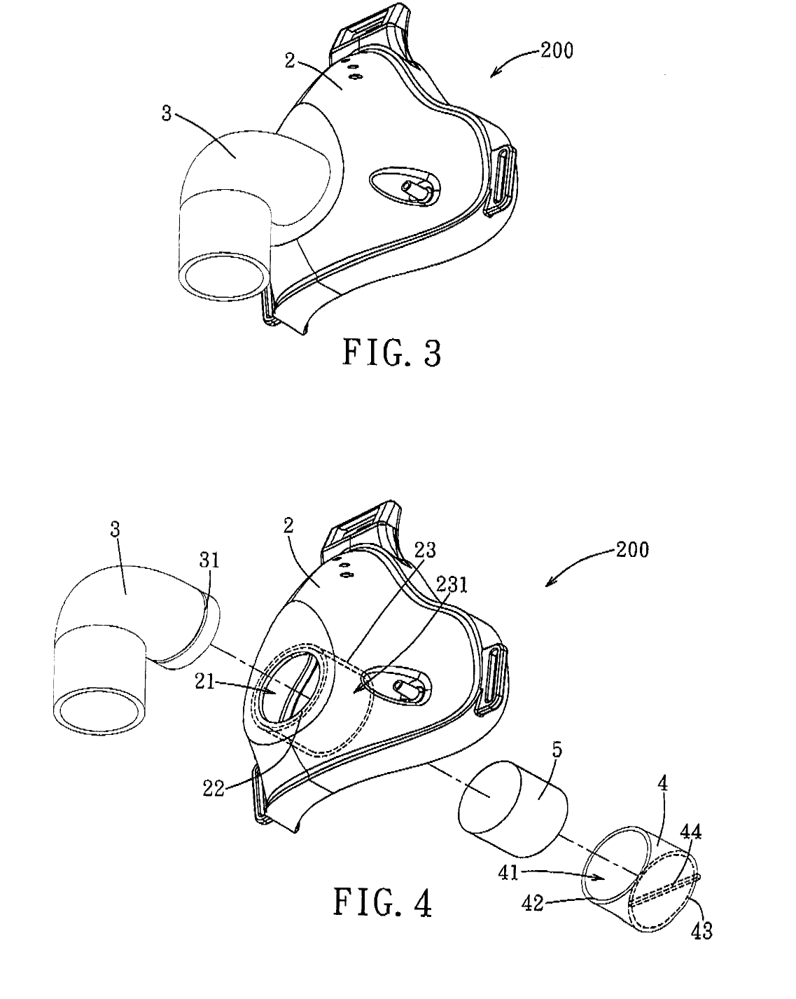

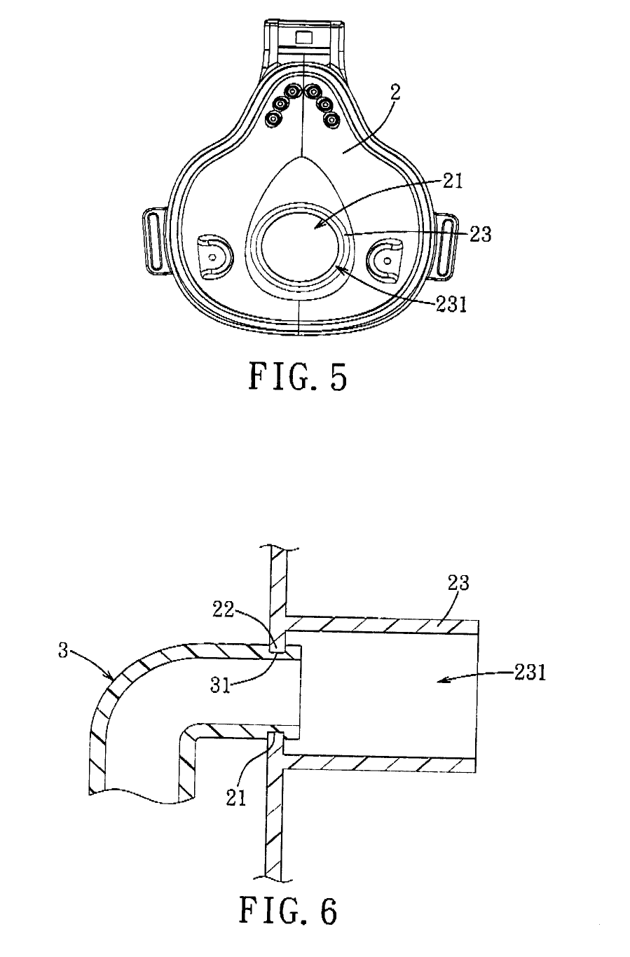

Referring to FIGS. 3 to 8, a respiratory mask 200 according to the first preferred embodiment of the present invention may be suitably used with a continuous positive airway pressure generator or any other flow generator that ventilates, and is shown to comprise a housing 2, an air supply tube 3, and a medium 5.

The housing 2 is adapted to cover the nose and mouth areas, and includes an inner surface, an air-passage opening 21, and a tubular wall 23 that projects inwardly from the inner surface and that defines a receiving hole 231 aligned and communicated with the air-passage opening 21. The receiving hole 231 has a diameter larger than that of the air-passage opening 21. A tubular sleeve 4 is inserted into the receiving hole 231, defines a receiving chamber 41, and has two opposite open ends 42, 43. A stop member 44, in the form of an elongated rod, is provided on the open end 43.

The medium 5 is disposed in the receiving chamber 41 via the open end 42, and is prevented from escaping the tubular sleeve 4 by the stop member 44. The medium 5 is made of a material that easily absorbs heat and moisture. In this embodiment, the medium 5 is made of a foamed, corrugated paper or treated material to enchance the collection of heat and moisture. Preferably, the foamed material is polyurethane. Alternatively, the medium 5 may also be made of a paper material.

The air supply tube 3 is connected between the gas flow generator and the housing 2. Air supplied by the gas flow generator can be inhaled by a patient through the air supply tube 3 and the housing 2. The air supply tube 3 has an annular engaging groove 31 on an outer surface thereof to engage a wall 22 that defines the air-passage opening 21. Through such a connection, the air supply tube 3 can be stably and fluidly connected to the housing 2.

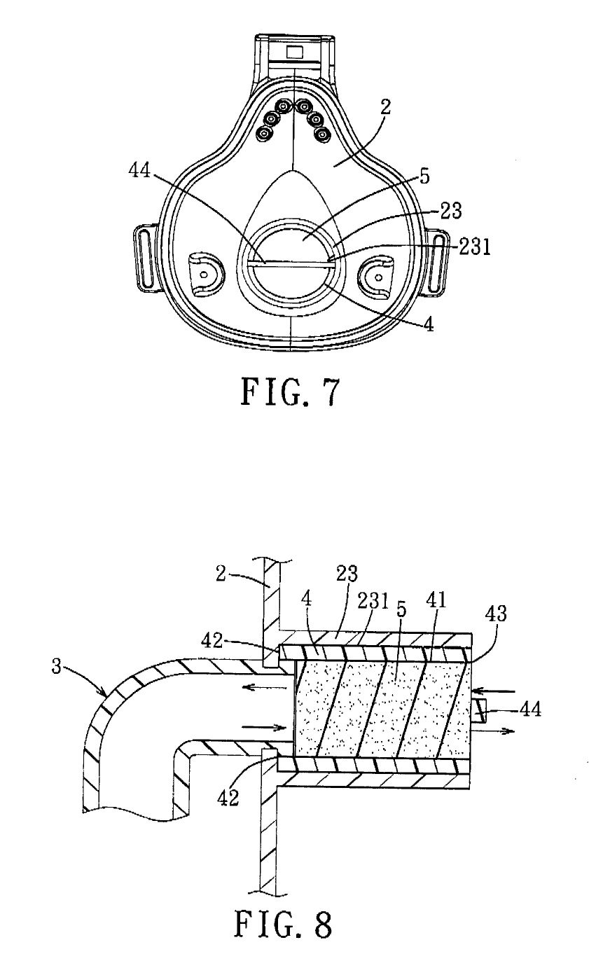

With reference to FIGS. 7 and 8, to assemble the medium 5 and the tubular sleeve 4 on the housing 2, the medium 5 is first disposed in the receiving chamber 41 of the tubular sleeve 4. Through the presence of the stop member 44, the medium 5 can be positioned within the receiving chamber 41. Afterwards, the tubular sleeve 4 is inserted into the receiving hole 231 of the tubular wall 23 through an inner end thereof so that the medium 5 corresponds in position with the air supply tube 3. This completes the assembly of the medium 5 and the tubular sleeve 4 on the housing 2. Preferably, the tubular sleeve 4 is interference fitted to the tubular wall 23 so that it does not easily separate from the tubular wall 23 when disposed in the receiving hole 231 of the tubular wall 23.

When the patient uses the respiratory mask 200, since the patient's mouth and nose are immediately adjacent to an outer end of the medium 5, and the tubular sleeve 4 has two opposite open ends 42, 43, gas exhaled by the patient can pass through the medium 5 and discharge through the air supply tube 3. When the gas passes through the medium 5, the medium 5 quickly absorbs and stores heat and moisture from the gas exhaled by the patient, thereby enhancing the effect of storing heat and moisture. Further, since the medium 5 is immediately adjacent to the patient's mouth and nose, the gas exhaled by the patient largely passes through the medium 5 and discharges through the air supply tube 3, so that creation of mist and condensation inside the housing 2 can be minimized during exhalation of the patient.

Moreover, while the patient is inhaling, as air supplied from the CPAP machine passes through the air supply tube 3 and the medium 5 which absorbs and stores heat and moisture, the air that passes through the medium 5 will have increased amount of heat and moisture for the patient to inhale. Through such a configuration, in a state where a heated humidifier is dispensed, air of suitable heat and moisture can be provided for the patient to inhale and to thereby reduce dryness in the patient's respiratory tract and the patient's resulting discomfort. Additionally, when an artificial nose and the heated humidifier are dispensed with, the structure of the whole respiratory system is more simple, and does not occupy a substantial space.

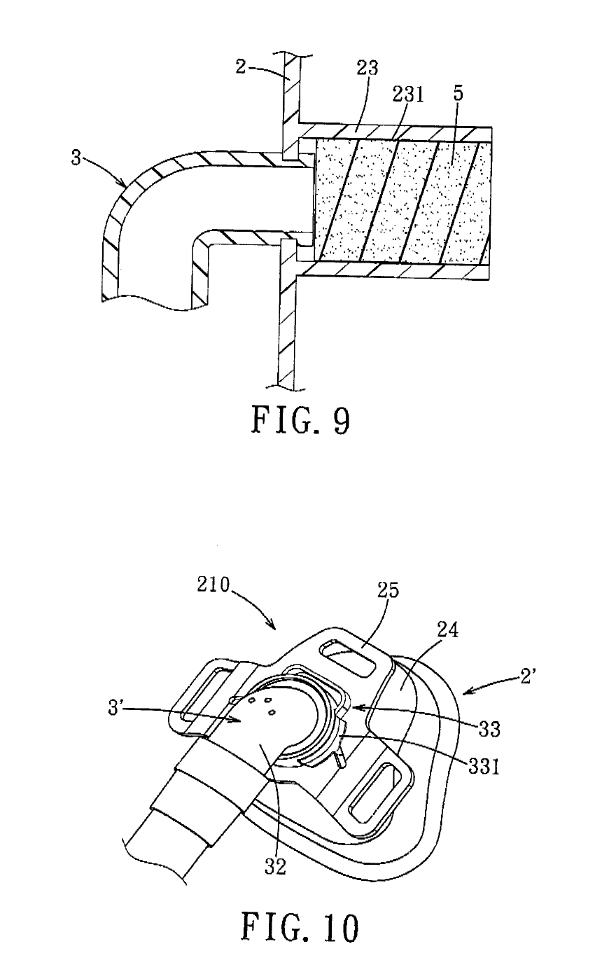

It is worth mentioning that, in an alternative embodiment, the medium 5 may be directly inserted into the receiving hole 231 of the tubular wall 23, as shown in FIG. 9, and the tubular sleeve 4 may be omitted. Preferably, an outer diameter of the medium 5 is larger than a hole diameter of the receiving hole 231, so that the medium 5 may be interference fitted to the tubular wall 23. Hence, the medium 5 cannot easily escape from the receiving hole 231 of the tubular wall 23.

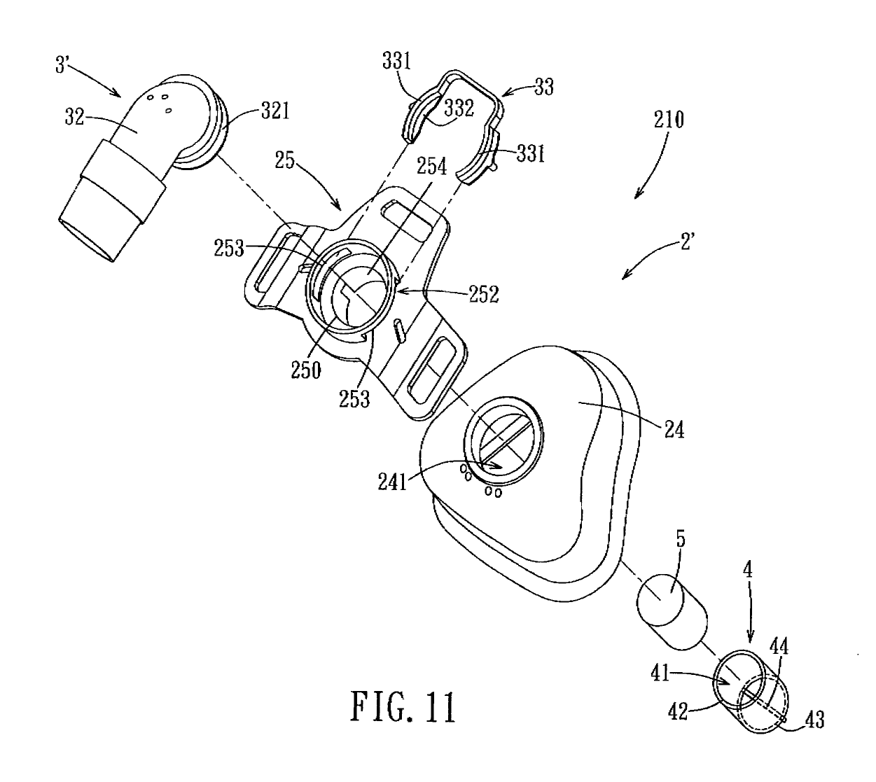

Referring to FIGS. 10 to 14, a respiratory mask 210 according to the second preferred embodiment of the present invention is shown to be similar to the first preferred embodiment. The difference between the first and second preferred embodiments resides in the structure of the housing 2′ and the air supply tube 3′.

The housing 2′ includes a mask body 24 having the air-passage opening 241, and a shroud 25 covering an outer surface of the mask body 24 for connection with a head strap (not shown). The shroud 25 has a shroud opening 250 aligned with the air-passage opening 241, a tubular wall 254 defining a receiving hole 255 and projecting from the shroud 25 into the mask body 24 through the air-passage opening 241, and an annular mounting flange 252 extending around the shroud opening 250 and projecting from the shroud 25 opposite to the mask body 24. Preferably, the tubular wall 254 is interference fitted to the mask body 24, so that the shroud 25 can be stably fixed to the mask body 24. The mounting flange 252 is formed with two opposite side slots 253.

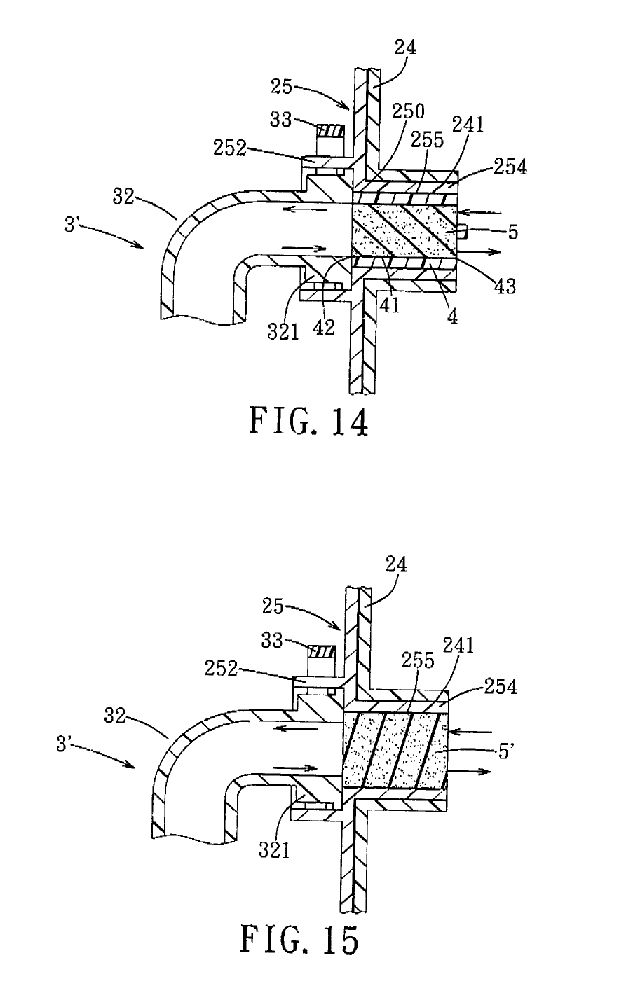

A tube body 32 of the air supply tube 3′ has an annular protrusion 321 projecting from an outer peripheral surface thereof and inserted into the mounting flange 252, as best shown in FIG. 14.

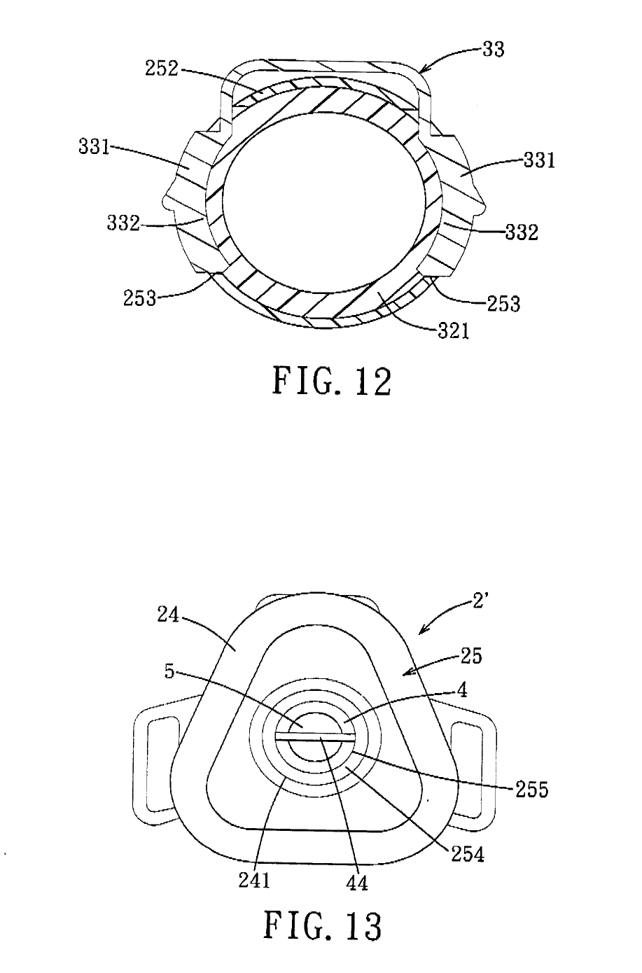

An inverted U-shaped retainer 33 has two opposite curved retaining arms 331 clamping therebetween an outer surface of the mounting flange 252, and two curved ribs 332 (see FIG. 12) projecting respectively from inner sides of the retaining arms 331. The curved ribs 332 are engaged to the annular protrusion 321 of the air supply tube 3′ at two opposite sides thereof, respectively, after passing through the respective side slots 253. Through such a connection, the tube body 32 of the air supply tube 3′ can be stably fixed to the mounting flange 252 of the shroud 25.

The tubular sleeve 4 containing the medium 5 is inserted into the receiving hole 255 of the tubular wall 254 through a rear end thereof, so that the medium 5 corresponds in position with the tube body 32 of the air supply tube 3′. Preferably, the tubular sleeve 4 is interference fitted to the tubular wall 254.

Alternatively, the medium 5′ may be directly inserted into the receiving hole 255 of the tubular wall 254, as shown in FIG. 15, and the tubular sleeve 4 may be omitted. Preferably, an outer diameter of the medium 5′ is larger than the hole diameter of the receiving hole 255, so that the medium 5′ may be interference fitted to the tubular wall 254.

From the aforesaid description, the respiratory mask 200, 210 in the two embodiments of the present invention has the medium 5, 5′ disposed in the housing 2, 2′ immediately adjacent to the patient's mouth and nose, so that the medium 5, 5′ can quickly absorb and store the heat and moisture of the gas exhaled by the patient, thereby enhancing the effect of storing good heat and moisture. Further, since the gas exhaled by the patient largely passes through the medium 5, 5′ and discharges through the air supply tube 3, 3′, the creation of mist and condensation inside the housing 2, 2′ can be minimized during exhalation of the patient. Hence, the objects of the present invention can be realized.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

I claim:

1. A respiratory mask comprising:
a housing including an inner surface, an air-passage opening, and a tubular wall that projects inwardly from said inner surface and that defines a receiving hole aligned and communicated with said air-passage opening;
an air supply tube connected fluidly to said air-passage opening; and
a medium disposed in said receiving hole and made of a material that can absorb heat and moisture.

2. The respiratory mask of claim 1, wherein said medium is interference fitted to said tubular wall.

3. The respiratory mask of claim 1, further comprising a tubular sleeve inserted into said receiving hole and having two opposite open ends, said medium being disposed inside said tubular sleeve.

4. The respiratory mask of claim 3, wherein said tubular sleeve is interference fitted to said tubular wall.

5. The respiratory mask of claim 4, wherein said tubular sleeve includes a stop member provided at one of said two opposite open ends to prevent escape of said medium from said tubular sleeve.

6. The respiratory mask of claim 5, wherein said medium is made of paper.

7. The respiratory mask of claim 5, wherein said medium is made of a foamed material.

8. The respiratory mask of claim 7, wherein said foamed material is polyurethane.

9. The respiratory mask of claim 1, wherein said housing includes a mask body having said inner surface and said air-passage opening, and a shroud covering said mask body and opposite to said inner surface, said shroud being formed with said tubular wall, and having a shroud opening aligned with said air-passage opening, said tubular wall projecting from said shroud into said mask body through said air-passage opening.

10. The respiratory mask of claim 9, wherein said shroud further has an annular mounting flange extending around said shroud opening and opposite to said mask body, said air supply tube being inserted into said annular mounting flange.

11. The respiratory mask of claim 10, further comprising a retainer disposed on said annular mounting flange and engaging said air supply tube.
